(19) United States
(12) Patent Application Publication
(10) Pub. No.: US 2009/0118551 A1
(43) Pub. Date: May 7, 2009

(54) Integrated Separation and Preparation Process

(76) Inventors: Andre Buijs, Amsterdam (NL); Leslie Andrew Chewter, Amsterdam (NL); Gerrit Jan Harmsen, Amsterdam (NL); Evert Van Der Heide, Amsterdam (NL); Jean-Francois Menard, Amsterdam (NL); Dominicus Fredericus Mulder, Amsterdam (NL); Wouter Spiering, Amsterdam (NL)

Correspondence Address: SHELL OIL COMPANY, P O BOX 2463, HOUSTON, TX, 772522463, US

(21) Appl. No.: 11/794,448

(22) PCT Filed: Dec. 27, 2005
(86) PCT No.: PCT/EP2005/057173

§ 371 Date: May 2, 2008

(30) Foreign Application Priority Data
Dec. 31, 2004 (EP) 04107074.9

(51) Int. Cl. B01D 53/22; C07C 29/04; C07C 27/06; C07C 5/02; C07C 5/03

(52) U.S. Cl. 568/884; 96/372; 96/379; 422/129; 95/43; 568/895; 585/379; 585/700

(57) ABSTRACT

Integrated separation and preparation process comprising a gas separation process wherein a first component is separated from a mixture of components by diffusion of the first component through a porous partition into a stream of sweeping component; and a preparation process wherein the sweeping component is used as feed. Separation unit and device for use in such a process and industrial set-up for use in such a process.

### CROSS REFERENCE TO RELATED APPLICATION

This application claims priority to International Patent Application Number PCT/EP2005/057173 filed Dec. 27, 2005, the entire disclosure of which is herein incorporated by reference.

### FIELD OF THE INVENTION

This invention relates to an integrated separation and preparation process.

### BACKGROUND OF THE INVENTION

In chemical industry several separation techniques are available to separate two or more components in a gaseous mixture. Examples of such separation techniques are known in the art and can be found in e.g. chapter 5.7 of “Process Design Principles” by W. Seider et al., published by John Wiley & Sons, inc. 1999.

The most generally applied technique is distillation. A disadvantage of distillation techniques, however, is the large amount of energy that is consumed to establish the separation of those compounds in a mixture.

Another technique that can be used is membrane separation by gas permeation. Herein a gas mixture is compressed to a high pressure and brought into contact with a non-porous membrane. The permeate passes the membrane and is discharged at a low pressure whereas the retentate does not pass through the membrane and is maintained at the high pressure of the feed. Examples for such a membrane separation method are described in U.S. Pat. No. 5,435,836 and U.S. Pat. No. 6,395,243. In these processes involving a gas separation via a membrane, in order to pass through the membrane, the gas molecules need to interact with the membrane. This however requires the application of a high pressure differential over the membrane between the retentate and the permeate side of the membrane. Due to the pressure differences required, such membrane techniques still require a considerable amount of energy and costly equipment for maintenance of the pressure differential, for instance by vacuum, or pressure pumps, even if a high sweep flow volume and highly selective membranes are employed.

U.S. Pat. No. 1,496,757, dating from 1924, describes a process of separation gases which comprises diffusing the gases through a diffusion partition, removing the diffused gas away from the partition by means of a sweeping material and removing the sweeping material from the diffused gas. The process is said to operate on the principle of repeated fractional diffusion. This process differs from separation processes involving membranes as described above in the fact that no or hardly any pressure differential is present, while the mass transfer is controlled by frictional diffusion with a sweep gas component continuously added to one chamber and diffusing counter-currently through the porous partitioning layer. This process thus does not require the use of expensive selectively permeable membranes.

Recently, M. Geboers, in his article “FricDiff: A novel concept for the separation of azeotropic mixtures”, OSPT Process Technology, PhD projects in miniposter form, published by the National Research School in Process Technology OSPT (2003) page 139, described a process for separating an azeotropic vapour mixture of 2-propanol (IPA) and water by letting it inter-diffuse with CO₂. In a subsequent step separation of the 2-propanol and CO₂ proceeds via condensation.

A disadvantage of this process is the required separation of product from the CO₂ stream, and if applied on an industrial scale, the procurement of a large sweep gas stream.

The use of the described diffusion-based separation method can thus still be improved by integration with a preparation process. The subject invention therefore provides for an integrated separation and preparation process.

### SUMMARY OF THE INVENTION

Accordingly, the present invention provides an integrated separation and preparation process comprising a gas separation process wherein a first component is separated from a feed stream comprising a mixture of components by diffusion of the first component through a porous partition into a stream of sweeping component; and a preparation process wherein the sweeping component is used as feed.

By using the sweeping component in a subsequent reaction step, more effective use of this sweeping component is made and an advantageous integrated separation and preparation process is obtained. A “separate” sweeping component can be avoided, because a reactant in a subsequent preparation process can be used as sweeping component. Preferably, the pressure on both sides of the porous partition is essentially equal.

The process according to the invention is especially advantageous in a process wherein the mixture of components from which the first component is separated is an azeotropic mixture, in view of the extensive costs of conventional distillation techniques for separation of such an azeotropic mixture.

The invention furthermore provides a separation unit in which the above process can be carried out.

### BRIEF DESCRIPTION OF THE DRAWINGS

FIG. 1 is a schematic three-dimensional view of a separation unit according to the present invention

FIG. 2 is a schematic representation of a process and a set-up according to the invention.

FIG. 3 is a schematic process for the separation and preparation of an alkanol according to the invention.

FIG. 4 is a schematic process for the separation and preparation of an alkylene glycol according to the invention.

FIG. 5 is a plot of molar flow of isopropanol, water and propene in channels (1) and (2) of an ideal separation unit operated in counter-current flow as a function of axial distance along the separation.

### DETAILED DESCRIPTION OF THE INVENTION

By an integrated separation and preparation process is understood a process wherein one or more of the components involved in the separation process is also a component involved in the preparation process. In the process of the present invention, the component used in the separation process as a sweeping component is used as a feed component in the preparation process.

By a gas separation process is understood that during this separation process at least part of the first component, mixture of components and sweeping component is in the gaseous state during the separation process.

Preferably at least 50% wt of the first component, mixture of components and sweeping component is in the gaseous state, more preferably at least 80% wt, and even more preferably in the range from 90 to 100% wt is in the gaseous state. Most preferably all components are completely in a gaseous state during the separation process. A component which is normally in the liquid state under ambient temperature (25° C.) and pressure (1 bar) can be vaporized to the gaseous state, for example by increasing temperature or lowering pressure, before diffusing through the porous partition. The diffusion during the gas separation process is hence preferably gas diffusion.

Without wishing to be bound by any kind of theory, the diffusion of the first component through the porous partition during the separation process is thought to be based on the so-called principle of frictional diffusion. This frictional diffusion is believed to be due to a difference in the rate of diffusion of a one component compared to one or more other components. As explained also in U.S. Pat. No. 1,496,757, a component having a faster rate of diffusion will more quickly pass a porous partition than a component having a slower rate of diffusion. The quicker component can be removed by the stream of sweeping component, resulting in a separation of such a first, quicker component from the remaining components. In the above a quicker component is understood to be a component having a higher binary diffusion coefficient together with the sweeping component than a slower component.

By a sweeping component is understood a component which is able to sweep away a first component that has diffused through the porous partition. It can be any component known to the skilled person to be suitable for this purpose. Preferably a component is used which is at least partly gaseous at the temperature and pressure at which the separation process is carried out. More preferably a sweeping component is used which is nearly completely, and preferably completely gaseous at the temperature and pressure at which the separation process is carried out. For practical purposes the invention may frequently be carried whilst using a sweeping component having a boiling point at atmospheric pressure (1 bar) in the range from −200 to 500° C. More preferably a sweeping component is used which has a boiling point at atmospheric pressure (1 bar) in the range from −200 to 200° C. Examples of components that can be used as sweeping component include carbon monoxide, carbon dioxide, hydrogen, water, oxygen, oxides, nitrogen-containing compounds, alkanes, alkenes, alkanols, aromatics, ketones.

The mixture and the sweeping component are separated by a porous partition, through which the first component diffuses from the mixture into the stream of sweeping component.

The porous partition can be made of any porous material known to the skilled person to be suitable for use in a process where it is contacted with the reactants. The porous partition can be made of a porous material that assists in the separation of the components by for example adsorption or absorption effects, provided that the separation by diffusion prevails.

According to M Stanoevic, Review of membrane contactors designs and applications of different modules in industry, FME Transactions (2003) 31, 91-98, a membrane phase, which is set between two bulk phases, has the ability to control mass transfer between the two bulk phases in a membrane process. Contrary to such a membrane, the porous partitioning layer according to the subject invention is set between the two bulk phases, but has in principle no ability to control the mass transfer of any of the species involved. It does therefore essentially not interact with the species to be separated other than offering pores, but merely serves to avoid mixing of the two bulk phases, contrary to membrane separations.

The subject porous partition is thus essentially not a selectively permeable membrane. A membrane is a barrier that allows some compounds to pass through, while effectively hindering other compounds to pass through, thus a semi-permeable barrier of which the pass-through is determined by size or special nature of the compounds. Membranes used in gas separation techniques are for instance those disclosed in U.S. Pat. No. 5,843,209. Membranes selectively control mass transport between the phases or environments.

Contrary to such membranes, the porous partition is a barrier that allows the flow of all components, albeit at different relative rates of diffusion. Without wishing to be bound to any particular theory it is believed that in the porous partitioning, the mass transfer is controlled by frictional diffusion with a sweeping gas component continuously added to one chamber and leaving the other chamber and diffusing counter-currently through the porous partitioning layer.

Preferably the material used for the porous partition is essentially inert or inert to the components used in the separation process. In practice the invention may frequently be carried out whilst using filter cloth, metal, plastics, paper, sandbeds, zeolites, foams, or combinations thereof as material for the porous partition. Examples include expanded metals, e.g. expanded stainless steel, expanded copper, expanded iron; woven metals, e.g. woven copper, woven stainless steel; cotton, wool, linen; porous plastics, e.g. porous PP, PE or PS. In a preferred embodiment the porous partition is prepared from woven or expanded stainless steel.

The convective volumetric flow (m³/s) across the porous partition layer (assuming laminar or Poiseuille flow) is given by formula I:

$$Q = \frac{π Δ P ɛ {d}_{p}^{4}}{128 μ δ} \qquad ( I )$$

wherein ε represents the porosity (fraction of surface area covered by pores), dₚ represents the pore diameter, δ represents the thickness of the porous layer, and ΔP represents the pressure drop across the porous layer as well as the physical properties of the gas (viscosity and density).

Preferred porous materials should have a high porosity (e) to maximise the useful surface area. The preferred porous layers porous have a porosity of more than 0.5, preferably more than 0.9, yet more preferably more than 0.93.

The thickness of the porous layer is preferably as low as possible. Without wishing to be bound to any particular theory, it is believed that the diffusive rate is inversely proportional to the thickness of the porous layer, and thus the required surface area of the porous layer is proportional to the thickness.

The porous partition can vary widely in thickness and may for example vary from a partition having a thickness of 1 or more meters to a partition having a thickness of 1 or more nanometres. For practical purposes the invention may frequently be carried out using a porous partition having a thickness in the range from 0.0001 to 1000 millimetres, more preferably in the range from 0.01 to 100 millimetres, and still more preferably in the range from 0.1 to 10 millimetres. Preferred porous layers have a thickness in the range of from 0.5 to 1.5 millimetres, preferably in the range of from 0.8 to 1.2 millimetres, and more preferably in the range of from 0.9 to 1.1 millimetres.

The amount, size and shape of the pores used in the porous partition may vary widely. The shape of the pores used in the porous partition may be any shape known to the skilled person to be suitable for such a purpose. The pores can for example have a cross-section shaped as slits, squares, ovals or circles. Or the cross-section may have an irregular shape. For practical purposes the invention may frequently be carried out using pores having a cross-section in the shape of circles. The diameter of cross-section of the pores may vary widely. It is furthermore not necessary for all the pores to have the same diameter. For practical purposes the invention may frequently be carried out using pores having a cross-section “shortest” diameter in the range from 1 manometer to 10 millimetre. By the “shortest” diameter is understood the shortest distance within the cross-section of the pore. Preferably this diameter lies in the range from 20 nanometre to 2 millimetres, more preferably from 0.1 to 1000 micrometer, more preferably in the range from 10 to 100 micrometer.

Preferably, the pores (dₚ) in the material should be relatively small to prevent convective flow. The exact size and proportions depend on the thickness of the porous layer (α) and the pressure drop (ΔP) across the porous layer as well as the physical properties of the gas (viscosity and density).

Pores having a small diameter, e.g. in the range from 0.1 to 100 nanometres have the advantage that the control on pressure differences becomes more easy. Pores having a larger diameter, e.g. in the range from 100 to 1000 nanometres have the advantage that a better separation can be obtained. For instance at a pressure drop (ΔP) of around 10 Pa across the porous partition, the pores should have a diameter below 10 micrometer to prevent substantial convective flow as compared to the desired diffusive flow. At a pressure drop (ΔP) of 1 Pa, pores having a diameter of 30 micron should be preferred. However, pressure drop and pore diameter should be chosen in such way that a Knudsen diffusion regime is avoided.

Is it understood that the relative rates of diffusion through the porous layer of different gases are dependent on the relative magnitudes of their binary diffusion coefficients, and not or only to a lesser extent on the properties of the porous material.

The pores may furthermore vary widely in tortuosity, that is, they may vary widely in degree of crookedness. Preferably however, the pores are straight or essentially straight and have a tortuosity in the range from 1 to 5, more preferably in the range from 1 to 3.

The number of pores used in the porous partition may also vary widely. Preferably 1.0-99.9% of the total area of the porous partition is pore area, more preferably 40 to 99%, and even more preferably 70 to 95% of the total area of the partition is pore area. By pore area is understood the total surface area of the pores. For practical purposes the invention may frequently be carried out using a number of pores and a pore size such that the ratio of total surface area of pores in the partition to the gas volume of the mixture of components lies in the range from 0.01 to 100,000 m²/m³, preferably in the range from 1 to 1000 m²/m³.

The length of the porous partition in the direction of the flow of the stream of sweeping component may also vary widely. When the length of the layer is increased both building costs of the separation as well as the extent of separation increase. For practical purposes the invention may frequently be carried out using a porous partition having a length along the flow-direction of the sweeping component in the range from 0.01 to 500 meters, more preferably in the range from 0.1 to 10 meters.

The residence time of the sweeping component and/or the mixture of components in the separation unit can vary widely. For practical purposes the invention may frequently be carried out using a residence time for sweeping component and/or the mixture of components in the separation unit in the range from 1 minute to 5 hour. Preferably a residence time is used in the range from 0.5 to 1.5 hours.

The velocity of the sweeping component used in the process of the invention may vary widely. For practical purposes the invention may frequently be carried out at a velocity of the sweeping component in the range from 1 to 10,000 meters/hour, preferably in the range from 3 to 3000 meters/hour and more preferably in the range from 10 to 1000 meters/hour. If not stationary, similar velocities can be used for the mixture of components.

The flux of the diffusion of the first component through the porous partition can vary widely. For practical purposes the invention may frequently be carried out at a diffusion flux of the first component through the porous partition in the range from 0.03 to 30 kg/m²/hour, preferably in the range from 0.1 to 10 kg/m²/hour and more preferably in the range from 0.5 to 1.5 kg/m²/hr.

For practical purposes the invention may frequently be carried out by removing from 10 to 100% wt of the first component, based on the total amount of first component present in the mixture of components when starting the separation process, from the mixture of components. More preferably at least 30% wt, and more preferably at least 50% wt of first component present in the mixture is removed from the mixture of components during the separation process. Even more preferably in the range from 70 to 100% wt of first component, based on the total amount of first component present in the mixture of components when starting the separation process, is removed from the mixture of components during the separation process. Especially when removing a high percentage, e.g. in the range from 70 to 100% wt, of first component from the mixture of components, other components might also diffuse from the mixture of components into the stream of sweeping component. When such other components co-diffuse, they can be removed in an additional intermediate step before entering the preparation process; or, alternatively, such other co-diffused components can remain in admixture with the sweeping component and/or with the diffused first component during a subsequent preparation process. Possibly such other co-diffused components can be removed via a bleed stream in such a subsequent preparation process.

In another embodiment the separation process according to the invention can be combined with an additional separation process, including conventional distillation and/or membrane separation. The additional separation process can for example be used for removing other co-diffused components from the mixture of sweeping component and first component, or it can be used to remove other components from the mixture of components, before or after removal of the first component. Furthermore an additional separation process can be used to further remove first component from a mixture of components from which at least part of the first component has already been removed.

The first component can be separated from a stationary mixture by diffusion through a porous partition into a stream of sweeping component. Preferably, however, a separation process is used, wherein the first component is separated from a stream of a mixture of components on one side of a porous partition, by diffusion through such porous partition, into a stream of sweeping component on the on the opposite side of the porous partition. Such a separation process might be carried out co-currently, counter-currently or cross-currently. Preferably, however, such a separation process is carried out whilst having a stream of the mixture of components and a stream of sweeping component flowing counter-currently in respect of each other. The separation process can be carried out continuously, semi-batch or batch-wise. Preferably the separation process is carried out continuously.

The flow velocity of the stream of sweeping component can vary widely. For practical purposes the invention may frequently be carried out using a flow velocity for the stream of sweeping component in the range from 0.01 to 300 kmol/hour, more preferably in the range from 0.1 to 100 kmol/hour. The flow velocity of any flow of mixture of components (if not stationary) can also vary widely. For practical purposes the invention may frequently be carried out using a flow velocity for the stream of sweeping component in the range from 0.01 to 300 kmol/hour, more preferably in the range from 0.1 to 100 kmol/hour.

The temperature applied during the separation process can vary widely. Preferably such a temperature is chosen such that all components are completely gaseous during the diffusion process. More preferably the temperature in the separation process is the same as the temperature in the preparation process. For practical purposes the invention may frequently be carried out using a temperature in the range from 0 to 500° C., preferably in the range from 0 to 250° C. and more preferably in the range from 15 to 200° C.

The pressures applied may vary widely. Preferably such a pressure is chosen that all components are completely gaseous during the diffusion process. More preferably the pressure in the separation process is the same as the pressure in the preparation process. For practical purposes the invention may frequently be carried out using a pressure in the range from 0.01 to 200 bar (1×10³ to 200×10⁵ Pa), preferably in the range 0.1 to 50 bar. For example the separation process may be carried out at atmospheric (1 atm., i.e. 1.01325 bar) pressure.

Independently from the overall pressures applied, the pressure difference over the porous partition is maintained as small as possible, e.g. in the range of 0.0001 to 0.1 bar, provided that separation by diffusion prevails over any separation due to mass motion because of large pressure differences. The pressure difference preferably is in the range of from 0.0001 to 0.01 bar, more preferably in the range of 0.0001 to 0.001 bar, yet more preferably in the range 0.0001 to 0.0001 bar, and most preferably in the range of from 0.0001 to 0.0005 bar. Hence, the pressure on both sides of the porous partition is considered nearly equal or essentially equal.

This may preferably be achieved by adding a pressure balancing means into the system, for instance by providing a flexible diaphragm that allows pressure peaks in one of the two fluid streams to pass on to the other. The separation process may be carried out in any apparatus known to the skilled person to be suitable for this purpose. For example separation units may be used such as the ones exemplified in U.S. Pat. No. 1,496,757. Preferably a separation unit, suitable for separating a first component from a mixture of components by diffusion of the first component through a porous partition into a stream of sweeping component, is used which separation unit comprises
a first chamber;
a second chamber, separated from the first chamber by a porous partition;
a first inlet for conveying a mixture of components to the first chamber;
a first outlet for discharging the remainder of the mixture of components after at least part of the first component has been removed from the first chamber;
a second inlet for conveying a sweeping component into the second chamber;
a second outlet for discharging a mixture of sweeping component and diffused first component from the second chamber.

The first and second chamber can be arranged in several ways. In a preferred embodiment one chamber is formed by the inside space of a tube and the other chamber is formed by a, preferably annular, space surrounding such tube.

Such an embodiment is considered to be novel and hence the present invention further provides a separation unit, suitable for separating a first component from a mixture of components by diffusion of the first component through a porous partition into a stream of sweeping component, which separation unit comprises

an outer tube; and

an inner tube, which inner tube has a porous wall, and which inner tube is arranged within the outer tube, such that a first space is present within the inner tube and a second space is present between the outer surface of the inner tube and the inner surface of the outer tube; and

a first inlet for conveying fluid into the first space and

a first outlet for discharging fluid from the first space; and

a second inlet for conveying fluid into the second space and

a second outlet for discharging fluid from the second space.

In a different preferred embodiment, the first and the second chamber are separated by a porous partition formed by stacks of plates or sheets of the porous material. In these stacks, at least two plates, i.e. an upper plate and a lower plate comprising the porous partition material are layered above each other in such way as to provide an intermediate compartment, which is blocked off at one end, while fluidly connected to an open space at the other end. In stacks comprising more than two layers, the openings on adjacent sides of each intermediate compartment are blocked alternately. Hence, the stack separates a first chamber and a second chamber as set out above, while the chambers are at least in part formed by the stack. The plates of comprising the porous partition material may be at any suitable shape, for instance rectangular; they may be of even shape and size, or uneven. The latter is preferred since then one side of a plate is longer than the other side, and thus the flow of the faster flowing gas passes across the shorter distance, thereby lowering the pressure drop.

The compartments are typically defined by spacers or structures that are offset and support the porous partition. The spacer, along with the porous partition material connected thereto defines the intermediate compartment which may serves as retentate or sweeping compartment. The pressure drop may also conveniently be adjusted by using different spacers for the sweep gas and feed gas compartments.

Adjacent compartments have the porous partition positioned there-between in the shape of layered plate-like or sheet-like structures, thereby providing a flow path for both fluid streams with a large surface. The assembly of retentate and sweeping compartments may be in alternating order or in any of various arrangements necessary to satisfy design and performance requirements. The stack arrangement is typically bordered by a seal at one end and a fluid connection to another compartment at an opposite end.

The compartments are suitably placed into a separator vessel such that they are fluidly connected either to a fluid stream, while they are sealed towards the respective opposite fluid stream, thus separating the two fluid feed streams. The feeds of the two fluid streams are fed preferably in a cross flow arrangement to the alternate sides of the separator vessel, i.e. to arrive at perpendicular flow or cross-flow direction towards each other. This serves to bring the flows out of line (i.e. not co-linear flows) so that they can be fed to the vessels fluid inlet and outlet openings more easily.

The separation device suitable comprises a vessel comprising a first fluid inlet opening positioned proximate to a side of the vessel and a first fluid outlet opening positioned proximate to an opposing side of the vessel; a second fluid inlet opening positioned proximate to a side of the vessel and a second fluid outlet opening positioned proximate to an opposing side of the vessel, wherein the first and second inlets and outlets respectively are position in such way, that the flow direction of a first fluid stream entering the vessel at the first inlet, and leaving it at the first outlet, and a second fluid stream entering the vessel at the second inlet, and leaving it at the second outlet are essentially perpendicular to each other; and wherein the porous partition between the two fluids comprises a stack of plate-like structures which are sealed toward the first fluid stream, while fluidly connected to the second fluid stream, thereby forming an exterior flow space for the first stream defined at least partially by and positioned at least partially between an upper plate and a lower plate of porous material, and an interior flow space for the second stream, defined at least partially by and positioned at least partially between the opposite sides of the upper plate and the lower plate to prevent fluid flow from the exterior flow space into the interior flow space. The advantage of using a stacked separation device is that in cross-flow many parallel compartments are alternately connected to the feed stream and to the sweep gas stream, thus providing for a large surface in a relatively compact arrangement.

The fluids are, each independently, for preferably at least 50% wt in the gaseous state, more preferably at least 80% wt, and even more preferably in the range from 90 to 100% wt. Most preferably the fluids are nearly completely or completely gaseous.

Furthermore the inner tube and the outer tube are preferably arranged essentially co-axially.

The first space can either be used as a first chamber or as a second chamber and the second space can respectively be used as a second chamber or as a first chamber. Both the first as well as the second space can have multiple inlets and outlets. Preferably the first space present within the inner tube has only one inlet and only one outlet. The second space preferably has two or more, preferably 2 to 100 inlets and/or outlets or an inlet and/or outlet in the shape of a circular slit.

The inner tube can be arranged substantially eccentrically within the outer tube such that the central axis of the inner tube is arranged substantially parallel to the central axis of the outer tube. Preferably, however the inner tube is arranged substantially concentrically within the outer tube such that the central axis of the inner tube substantially coincides with the central axis of the outer tube.

The cross-section of the tubes can have any shape known to the skilled person to be suitable. For example, the tubes can independently of each other have a cross-section in the shape of a square, rectangle, circle or oval. Preferably the cross-section of the tubes is essentially circular.

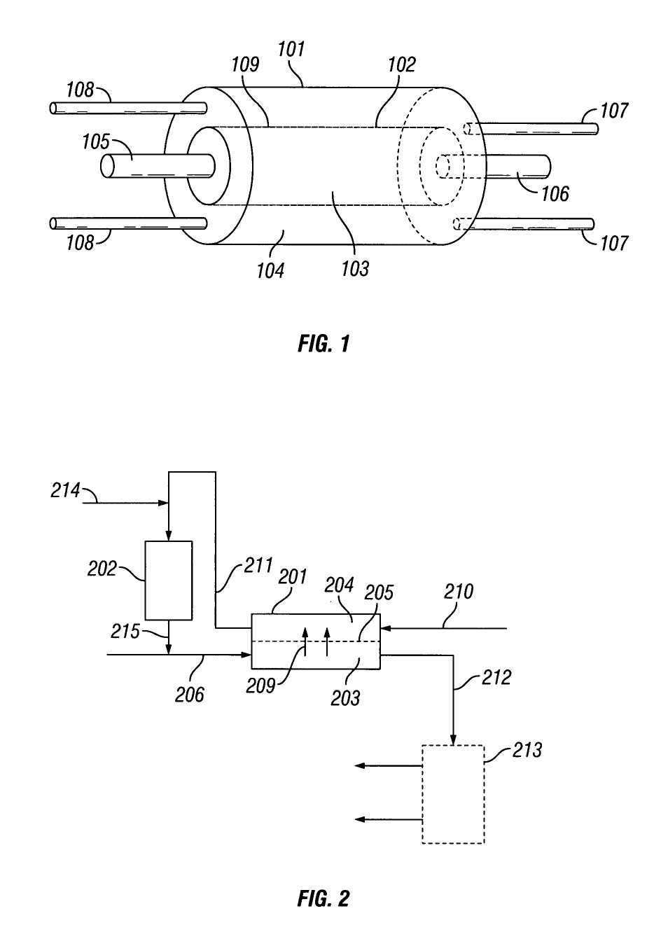

The invention will be described by way of example with reference to FIG. 1. FIG. 1 is a schematic three-dimensional view of a separation unit according to the present invention. FIG. 1 illustrates a separation unit having an outer tube (101) and an inner tube (102), which inner tube is co-axially arranged within the outer tube, such that

a first space (103) is present within the inner tube (102) and

a second space (104) is present between the outer surface of the inner tube (102) and the inner surface of the outer tube (101); and

comprising an inlet (105) into the first space and an outlet (106) from the first space; and an inlet (107) into the second space and an outlet (108) from the second space;

which inner tube has a porous wall (109).

In a further preferred embodiment the separation process is carried out in a separation device comprising a multiple of separation units, preferably in the range from 2 to 100,000, more preferably in the range from 100 to 10,000 separation units per separation device. Such a separation device is considered to be novel and therefore the present invention furthermore provides a separation device comprising two or more separation units, suitable for separating a first component from a mixture of components by diffusion of the first component through a porous partition into a stream of sweeping component, wherein each separation unit can comprise

a first chamber;

a second chamber, separated from the first chamber by a porous partition;

a first inlet for conveying a mixture of components to the first chamber;

a first outlet for discharging the remainder of the mixture of components after at least part of the first component has been removed from the first chamber;

a second inlet for conveying a sweeping component into the second chamber;

a second outlet for discharging a mixture of sweeping component and diffused first component from the second chamber.

The separation units can be arranged in the separation device in any manner known to suitable for this purpose by the skilled person. Preferably the separation units are arranged sequentially or parallel to each other in the separation device. The separation units can for example be sequentially arranged in an array. If such an array of sequentially arranged separation units is used, any pressure loss on either one side is preferably compensated by a intermediate stream of respectively mixture of components or sweeping component.

In an advantageous embodiment, the first or second chambers of two or more separation units are blended together such that two or more separation units share the same first or second chamber.

For example the present invention provides a multitubular separation device comprising

a substantially vertically extending vessel,

a plurality of tubes having a porous wall, arranged in the vessel parallel to its central longitudinal axis of which the upper ends of the tubes are fixed to an upper tube plate and in fluid communication with a top fluid chamber above the upper tube plate and of which the lower ends are fixed to a lower tube plate and in fluid communication with a bottom fluid chamber below the lower tube plate,

supply means for supplying a first fluid to the top fluid chamber and

an effluent outlet arranged in the bottom fluid chamber;

supply means for supplying a second fluid to the space between the upper tube plate, the lower tube plate, the outer surface of the tubes and the vessel wall and

an effluent outlet from such space between the outer surface of the tubes and the vessel wall.

The fluids are, each independently, for preferably at least 50% wt in the gaseous state, more preferably at least 80% wt, and even more preferably in the range from 90 to 100% wt. Most preferably the fluids are nearly completely or completely gaseous.

A mixture of components can for example be supplied to the space inside the tubes or to the space between the outer surface of the tubes and the inner surface of the vessel wall; and the sweeping gas can be supplied to respectively the space between the outer surface of the tubes and the inner surface of the vessel wall or the space inside the tubes.

In the preparation process the sweeping component can be reacted in one or more steps to obtain a product. The product can be a final product, but can also be an intermediate product which needs to be reacted further. In addition to such an intermediate or final product, or a combination thereof, one or more by-products might be prepared. By reacting is understood that the sweeping component is chemically changed. For example, the sweeping component can be chemically split into two or more separate products or the sweeping component can be reacted with one or more other components into one or more products. Examples of possible reactions include but are not limited to hydration, dehydration, hydrogenation and dehydrogenation, oxygenation, hydrolysis, esterification, amination, carbonation, carbonylation, carboxylation, desulfurisation, deamination, condensation, addition, polymerisation, substitution, elimination, rearrangement, disproportionation, acid-base, telomerisation, isomerisation, halogenation, dehalogenation and nitration reactions. The reaction conditions applied can vary widely and can be those known to the skilled person to be suitable for such reaction. In practice, the invention may frequently be carried out at a temperature in the range from −100 to 500° C., more preferably in the range from 0 to 300° C., and at a pressure in the range of 0.01 to 200 bar, more preferably in the range of 0.1 to 50 bar. Any type of reactor known by the skilled person to be suitable for a reaction can be used. Examples of types of reactors include a continuously stirred reactor, slurry reactor or tube reactor.

One or more of reactions in the preparation process can optionally be carried out in the presence of a catalyst. Any catalyst known to the skilled person to be suitable for a specific reaction applied can be used. Such a catalyst can be homogeneous or heterogeneous and might for example be present in solution, slurry or in a fixed bed. The catalyst can be removed in a separate unit.

The diffused first component or co-diffused other components can optionally also be used in the preparation process. For example the diffused first component can be reacted with the sweeping component to prepare a product. Or, the diffused first component can be used to prepare an intermediate product, which is subsequently reacted with the sweeping component to prepare a further product. Or, the diffused first component can be used to be reacted with an intermediate product, which intermediate product was obtained from a reaction of the sweeping component, to obtain a further product. By diffused component is understood a component diffused from the mixture of components into the sweeping component during separation process.

The steps in the process of the invention can each be carried out in a continuous, semi-batch or batch manner. For example the separation process can be carried out in a continuous or semi-batch manner whereas the subsequent preparation process can be carried out in a batch manner. In a preferred embodiment, all steps are carried out in a continuous manner. Hence the present invention also provides a process according to the invention wherein this process is continuous.

The sweeping component can be forwarded directly or indirectly from the separation process as a feed to the preparation process. For example, other components, such as diffused first component present in admixture with the sweeping component after leaving the separation process, can be removed in an intermediate step. Separation of such components from such sweeping component can be carried out by any process known to the skilled person to be suitable therefore. For example distillation, flashing, precipitation or gas-liquid separation can be used. Preferably the sweeping component is forwarded directly from the separation into the preparation process or an intermediate step is only included for removing one or more diffused components. More preferably a mixture of the diffused first component and a diffused component is used in the preparation process.

The integrated separation and preparation process is preferably carried out in an industrial set-up comprising

a separation device comprising one or more separation units suitable for separating a first component from a mixture of components by diffusion of the first component through a porous partition into a stream of sweeping component, comprising one or more first chambers, one or more second chambers, separated from the first chamber or chambers by a porous partition, one or more inlets and one or more outlets,

one or more reactors comprising one or more inlets and one or more outlets, wherein the outlet of one or more separation units is connected directly or indirectly to one or more inlets of one or more reactors.

A process and set-up of the invention will be described by way of example with reference to FIG. 2. FIG. 2 is a schematic representation of a process and a set-up according to the invention.

FIG. 2 shows a separation unit (201) and a reactor (202). The separation unit comprises a first chamber (203) and a second chamber (204), separated from each other by a porous partition (205). A stream of a mixture of components (206) enters the separation unit (201) in a first chamber (203). A diffusion stream of first component (209) diffuses from the first chamber (203) into the second chamber (204), whilst a stream of sweeping component (210) is flowing in the second chamber (204) counter-currently to the stream of the mixture of components (206) in the first chamber (203). The diffusion stream of first component (209) is taken up by the sweeping component (210) to form a stream comprising a mixture of first component and sweeping component (211) leaving the separation unit. A stream of remainder of mixture of components (212), from which the first component has at least partly been removed, leaves the separation unit (201) to be optionally further purified in distillation train (213). The stream of mixture of first component and sweeping component (211) is transferred to reactor (202). If desired, additional first component can be added via an extra stream (214). The reactor (202) or extra stream of first component (214) can optionally comprise a homogeneous or heterogeneous catalyst (not shown). A stream of reaction mixture comprising product and first component (215) is recycled to the separation unit (201). Any homogeneous or heterogeneous catalyst can optionally be removed in a separate unit (not shown), before or after the separation unit (201).

Preferably the integrated separation and preparation process comprising the steps of

a) separating a first component from a mixture of components by diffusion of the first component through a porous partition into a stream of sweeping component, to obtain a mixture of first component and sweeping component;
b) optionally separating the mixture of first component and sweeping component obtained in step a) into first component and sweeping component;
c) using the sweeping component, optionally mixed with first component, as a feed to a reaction;
d) reacting the sweeping component in one or more steps to obtain a product.

In such a process step a) can be carried out as described hereinabove for the separation process and step d) can be carried out as described herein above for the preparation process.

In many cases the product obtained in step d) is present as part of a reaction mixture. Such a reaction mixture can be processed further to separate product, by-products and remainder of reactants. In an advantageous embodiment at least part of this reaction mixture is recycled to step a).

Hence, the present invention further provides separation and preparation process comprising the steps of

a) separating a first component from a mixture of components
by diffusion of the first component through a porous partition into a stream of sweeping component, to obtain a mixture of first component and sweeping component;
b) optionally separating the mixture of first component and sweeping component obtained in step a) into first component and sweeping component;
c) using the sweeping component, optionally mixed with first component, as a feed to a reaction;
d) reacting the sweeping component, and optionally the first component, in one or more steps to obtain a reaction mixture comprising a product;
e) recycling at least part of the reaction mixture to step a).

Such a process is especially advantageous when the first component is a reactant which is provided in surplus to the preparation process.

The process of the present invention is widely applicable.

For example the present invention provides a process as described above wherein the first component and the sweeping component are not separated in step b); a mixture of the first component and the sweeping component, is used as a feed to a reaction in step c); and the first component and the sweeping component are reacted with each other in step d).

Such a process can for example be used in a preferred embodiment for the preparation of an alkanol by hydration of an alkene, e.g. wherein the first component is water, the sweeping component is an alkene; and the first component and the sweeping component are reacted with each other in a hydration reaction to prepare an alkanol. When the water is used in surplus in the preparation process, at least part of a reaction mixture comprising water and alkanol can advantageously be recycled to the separation process of step a).

The alkanol preferably comprises from 2 to 10 carbon atoms. Examples of such alkanols include ethanol, n-propanol, isopropanol, n-butanol, isobutanol, pentanols and hexanols. Such alkanols can be prepared by reacting a corresponding alkene, having from 2 to 10 carbon atoms, with water. In addition, a mixture of alkanols can be prepared by reaction a corresponding mixture of alkenes. Preferred hydration reactions are those wherein propene is reacted with water to isopropanol; wherein butene is reacted with water into sec.-butanol; and wherein a mixture of propene and butene is reacted with water into a mixture of isopropanol and sec.-butanol.

Reaction conditions may vary widely. Any reaction conditions known by the persons skilled in the art to be suitable for reacting the alkene and water can be used. For example, both heterogeneous catalysts such as phosphoric acid on betonite clay or homogeneous catalysts such as sulphuric acid can be used.

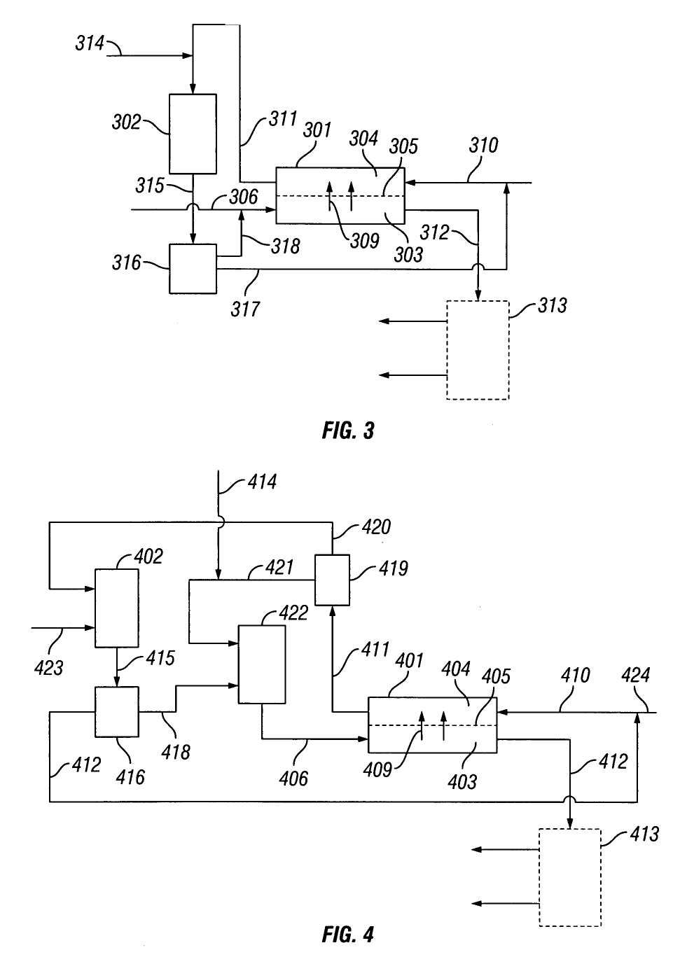

The obtained reaction mixture in step d) may contain a combination of alkanol and unreacted water. Such a reaction mixture can advantageously be recycled to step a). When the reaction mixture further comprises unreacted alkene, such a reaction mixture can still be recycled to step a). If desired, any unreacted alkene can also be separated from the alkanol product before separating the unreacted water or after separating the unreacted water in step a). Preferably any unreacted alkene in the reaction mixture is separated from the product alkanol before recycling the mixture of alkanol and water to the separation in step a), where after the mixture of alkanol and water is recycled to step a) as mixture of components and/or the separated alkene is recycled to step a) as sweeping gas. The removal of such unreacted alkene is preferably carried out by a partial flash condenser to recover alkene and crude alkanol product contaminated with water. An example of an alkanol separation and preparation process according to the invention is described by example with reference to FIG. 3. FIG. 3 is a schematic process for the separation and preparation of an alkanol according to the invention.

FIG. 3 shows a separation unit (301) and a reactor (302). The separation unit comprises a first chamber (303) and a second chamber (304), separated from each other by a porous partition (305). A stream of a mixture comprising alkanol and water (306) enters the separation unit (301) in a first chamber (303). A diffusion stream of water (309) diffuses from the first chamber (303) into the second chamber (304), whilst a stream of alkene sweeping component (310) is flowing in the second chamber (304) counter-currently to the stream of the alkanol and water (306) in the first chamber (303). The diffusion stream of water (309) is taken up by the stream of alkene (310) to form a stream comprising a mixture of alkene and water (311) leaving the separation unit. A stream of remainder of alkanol (312), from which the water has at least partly been removed, leaves the separation unit (301) to be optionally further purified in distillation train (313). The stream of mixture of water and alkene (311) is transferred to reactor (302). If desired, additional water can be added via an extra stream (314). The reactor (302) or extra stream of first component (314) can optionally comprise a homogeneous or heterogeneous catalyst (not shown). A stream of a reaction mixture comprising unreacted alkene, alkanol and unreacted water (315) is separated in a gas-liquid separator (316) into a stream of unreacted alkene (317) and a stream of mixture of water and alkanol (318). Both streams are recycled to the separation unit (301). Any catalyst is removed after the alkanol has left the separation unit.

The process of the invention can further be used in a further preferred embodiment for the preparation of an alkanol by hydrogenation of a ketone, e.g. wherein first component is hydrogen, the sweeping component is a ketone; and the first component and the sweeping component are reacted with each other in a hydrogenation reaction to prepare an alkanol. When the hydrogen is used in surplus in the preparation process, a mixture comprising hydrogen and alkanol can advantageously be recycled to the separation process of step a).

The alkanol preferably comprises from 2 to 10 carbon atoms. Examples of such alkanols include ethanol, n-propanol, isopropanol, n-butanol, isobutanol, pentanols and hexanols. Such alkanols can be prepared by reacting as the corresponding ketone, having from 2 to 10 carbon atoms with water. In addition, a mixture of alkanols can be prepared by reaction a corresponding mixture of ketones. Preferred hydrogenation reactions are those wherein dimethylketone (acetone) is reacted with hydrogen to isopropanol; wherein methylethylketone (2-butanon) is reacted with hydrogen into sec.-butanol; and wherein a mixture of dimethylketone and methylethylketone is reacted with hydrogen to a mixture of isopropanol and sec.-butanol.

Reaction conditions may vary widely, and can be those known to be suitable by the skilled person in the art.

The process can further be used in a further preferred embodiment for the hydrogenation of unsaturated compounds such as alkenes and aromatics, e.g. wherein the first component is hydrogen, the sweeping component is an alkene or an aromatic compound; and the first component and the sweeping component are reacted with each other to prepare an alkane. For example, benzene can be hydrogenated to cyclohexane, a useful intermediate in nylon synthesis. Reaction conditions may vary widely, and can be those known to be suitable by the skilled person in the art.

This invention further provides an integrated separation and preparation process wherein unreacted reactant is used as sweeping component to remove byproduct from a mixture of product and by-product. In a preferred embodiment such a process comprising the steps of

a) separating a byproduct from a mixture of product and byproduct by diffusion of the byproduct through a porous partition into a stream of reactant, to obtain a mixture of the byproduct and reactant;
b) optionally separating the mixture of byproduct and reactant obtained in step a) into byproduct and reactant.
c) using the reactant, optionally mixed with the byproduct, as a feed in a reaction;
d) reacting the reactant in one or more steps to obtain a mixture of product and by-product.

In a preferred embodiment, the byproduct is a byproduct which is prepared in a certain equilibrium with the product under reaction conditions. In such a case the by-product and reactant in step b) are preferably not separated and a mixture of reactant and byproduct is fed to the reaction in step c). Preferably a subsequent reaction mixture comprising product and by-product is recycled to step a).

In a further preferred embodiment the reactant is not fully reacted and the reaction mixture obtained in step d) comprises unreacted reactant, product and byproduct. Preferably such reaction mixture is separated into a stream of unreacted reactant and a stream of product and by-product, where after both streams are recycled to step a) and the unreacted reactant is used as sweeping component.

The above process can be advantageous to reduce the amount of byproduct made in a process.

In a further example the present invention provides such a process as described above wherein the first component and the sweeping component are separated in step b);

the separated sweeping component is used as a feed in a first reaction and the separated first component is used as a feed in a second reaction in step c); and the separated sweeping component is reacted in one or more steps to a product in step d).

The first component may be discarded or used in some other process. In a preferred embodiment, however, both the sweeping component as well as the first component are used in the preparation process of step d). For example, the separated sweeping component can be reacted in one or more steps with one or more other components to an intermediate product in step; and the intermediate product can be reacted with the separated first component in one or more steps to a subsequent product. Alternatively, the separated first sweeping component can be reacted in one or more steps with one or more other components to an intermediate product; and the intermediate product can be reacted with the separated sweeping component in one or more steps to a subsequent product.

Examples of such a process include a process for the preparation of an alkylene glycol comprising the steps of

a) separating water from a mixture of water and alkylene glycol by diffusion of the water through a porous partition into a stream of carbon dioxide, to obtain a mixture of the water and the carbon dioxide
b) separating the mixture of water and carbon dioxide obtained in step a) into water and carbon dioxide;
c) using the separated carbon dioxide as a feed in a first reaction and using the separated water as a feed in a second reaction;
d) reacting the separated carbon dioxide with an alkylene oxide in the first reaction to prepare an alkylene carbonate and reacting the alkylene carbonate with the separated water in a second reaction to prepare an alkylene glycol.

When the alkylene carbonate in step d) is reacted with a surplus of water to prepare a mixture of alkylene glycol and water, the mixture of alkylene glycol and water can advantageously be recycled to step a). When the reaction mixture of step d) furthermore contains unreacted carbon dioxide, such carbon dioxide can advantageously be separated from the alkylene glycol and water before the reaction mixture is recycled to step a), where after the carbon dioxide is separately recycled to step a) as a sweeping component.

The alkylene glycol preferably comprises from 2 to 10 carbon atoms. Examples of such alkylene glycols include monoethylene glycol (1,2-ethanediol) and monopropylene glycol (1,2-propanediol). Such alkylene glycols can be prepared by reacting the corresponding alkylene oxide comprising from 2 to 10 carbon atoms with carbon dioxide and water. Preferred reactions are those wherein monoethylene glycol is prepared from ethylene oxide, carbon dioxide and water and wherein monopropylene glycol is prepared from propylene oxide, carbon dioxide and water. Reaction conditions may vary widely, and can be those known to be suitable by the skilled person in the art.

An example of an alkylene glycol separation and preparation process according to the invention is described by example with reference to FIG. 4. FIG. 4 is a schematic process for the separation and preparation of an alkylene glycol according to the invention.

FIG. 4 shows a separation unit (401), a first reactor (402) and a second reactor (422). The separation unit comprises a first chamber (403) and a second chamber (404), separated from each other by a porous partition (405). A stream of a mixture comprising alkylene glycol and water (406) enters the separation unit (401) in a first chamber (403). A diffusion stream of water (409) diffuses from the first chamber (403) into the second chamber (404), whilst a stream of carbon dioxide sweeping component (410) is flowing in the second chamber (404) counter-currently to the stream of the alkylene glycol and water (406) in the first chamber (403). The diffusion stream of water (409) is taken up by the stream of carbon dioxide (410) to form a stream comprising a mixture of carbon dioxide and water (411) leaving the separation unit. A stream of remainder of alkylene glycol (412), from which the water has at least partly been removed, leaves the separation unit (401) to be optionally further purified in distillation train (413). The stream of mixture of water and carbon dioxide (411) is transferred to gas-liquid separator (419). Hereafter a stream of separated carbon dioxide (420) is transferred to a first reactor (402), whereas a stream of separated water (421) is transferred to a second reactor (422). In addition a stream of propylene oxide (423) is added to the first reactor (402). If desired, additional water can be added via an extra stream (414). The reactors (402 and 422), the stream of propylene oxide (423) or extra stream of first component (414) or an additional stream (not shown) can optionally be used to add homogeneous or heterogeneous catalyst (not shown). A stream of a reaction mixture comprising alkylene carbonate and unreacted carbon dioxide (415) leaving the first reactor (402) is separated in a gas-liquid separator (416) into a stream of carbon dioxide (417) and a stream comprising alkylene carbonate (418). The carbon dioxide is recycled to the separation unit (401) as a stream of carbon dioxide sweeping component (410). Possibly additional (make-up) carbon dioxide is added via an additional stream (424). The stream of alkylene carbonate (418) is added to the second reactor (422) where it is reacted with the stream of water (421). A stream of reaction mixture comprising water and alkylene glycol (406) is recycled to the separation unit (401). Optionally catalyst is removed after the alkylene glycol has left the separation unit or in between the reactors.

An example of a process wherein the first component and the sweeping component are separated in step b); the separated sweeping component is used as a feed to a reaction in step c); and the separated sweeping component is reacted in a dehydrogenation reaction in step d) whereas the separated first component is not reacted in such a step can be given by a process for the preparation of ketones.

The present invention hence also provides a process comprising the steps of

a) separating hydrogen from a mixture of hydrogen and ketone by diffusion of the hydrogen through a porous partition into a stream of alkanol, to obtain a mixture of the hydrogen and the alkanol;
b) separating the hydrogen and the alkanol;
c) using the separated alkanol as a feed in a reaction;
d) reacting the separated alkanol in a dehydrogenation to obtain a mixture of hydrogen and ketone. Advantageously such a mixture can be recycled to step a) to separate hydrogen from the ketone product.

The alkanol preferably comprises from 2 to 10 carbon atoms. Examples of such alkanols include ethanol, n-propanol, isopropanol, n-butanol, isobutanol, pentanols and hexanols. Such alkanols can be dehydrogenated to the corresponding ketone, having from 2 to 10 carbon atoms with water. Similarly mixtures of ketones can be prepared by dehydrogenation of corresponding mixtures of alkanols. Preferred dehydrogenation reactions are those wherein dimethylketone (acetone) is prepared from isopropanol; wherein methylethylketone (2-butanon) is prepared from sec.-butanol; and wherein a mixture of dimethylketone and methylethylketone is prepared from a mixture of isopropanol and sec.-butanol.

Reaction conditions may vary widely, and can be those known to be suitable by the skilled person in the art.

The invention will be illustrated by the following non-limiting examples.

### EXAMPLE 1

### Hydration of Propene to Prepare Isopropanol

Isopropanol can be obtained by hydration of propene in the presence of an acid catalyst. The main product, isopropanol, however, forms an azeotrope with water at 80.3° C.

In a first example a computer simulation is made for the separation of a mixture of water and isopropanol with help of propene as sweeping component. The multi-component gas-phase system is modelled using the Stefan-Maxwell approach to mass transfer. An assumption was made that the pores of the porous medium are so large that the gas-wall interactions can be neglected compared to the friction between the different gas particles.

The simulation was carried out for a separation unit having the following specifics:

a length (L) of 3 meter;
a total surface area of pores in the porous partition to gas volume of the mixture of isopropanol and water (a) of 100 m²/m³;
a temperature (T) of 35° C.;
a pressure (P) of 1 atmosphere (i.e. equivalent to 1 bar);
a porous partition thickness (δ) of 0.0861 meter; and the following binary diffusion coefficients

D_{H2O,IPA}=3.38*10⁻⁷ m²/s; D_{H2O,C3=}=1.06*10⁻⁶ m²/s; D_{IPA,C3=}=2.43*10⁻⁷ m²/s.

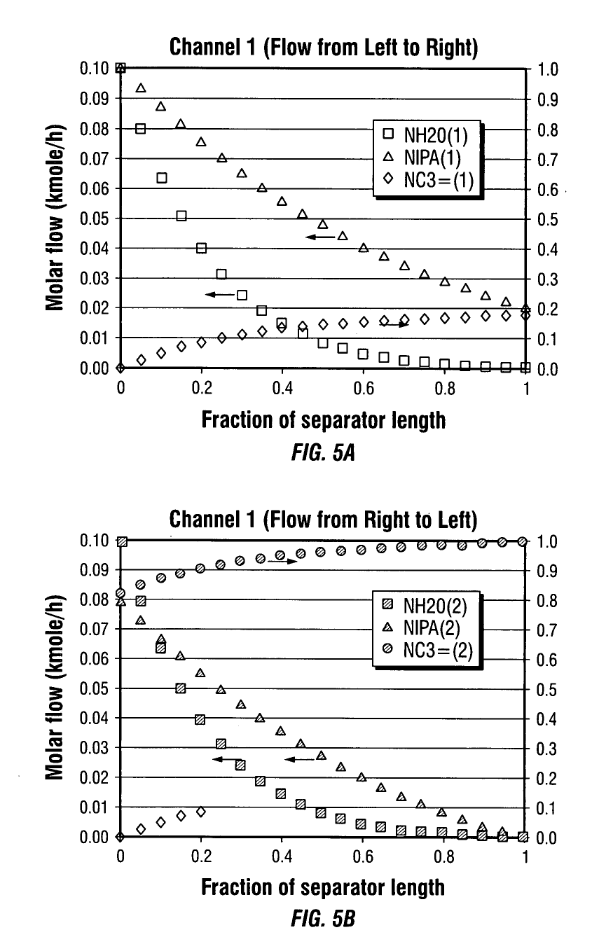

FIG. 5 shows a plot of molar flow of IPA, water and propene in channels (1) and (2) of an ideal separation device operated in counter-current flow as a function of axial distance along the separation. Flow in channel 1 is from left to right; flow in channel 2 is from right to left.

The extent of separation can be represented by RD, which is the ratio of the binary diffusion coefficient of the second reactant and the sweeping component to the binary diffusion coefficient of the product and the sweeping component, i.e.

RD=D_{2nd reactant, sweeping component}/D_{product, sweeping component}

For the above example the RD can be calculated to be

D_{H2O,C3=}/D_{IPA,C3=}=1.06*10⁻⁶/2.43*10⁻⁷=4.36

### COMPARATIVE EXAMPLE A AND EXAMPLES 2 AND 3

For several other hydration reactions of alkanols, at several temperatures and pressures the ratio of the binary diffusion coefficient of the first component and the sweeping component to the binary diffusion coefficient of the product and the sweeping component (RD) was calculated. The mixtures, sweeping components, temperatures, pressures and resulting RD are summarized in table 1.

As can be seen from comparing the results for comparative example A and example 2 in table 1, use of propene as an sweeping component provides even for a greater ratio between the binary diffusion coefficients (RD) than the carbon dioxide used by M. Geboers et al.

### EXAMPLE 4

Example 4 illustrates the ratio of the binary diffusion coefficient of the first component and the sweeping component to the binary diffusion coefficient of the product and the sweeping component (RD) for a dehydration reaction of sec-butanol to prepare methyl-ethylketone and hydrogen. The results are given in table 1.

### EXAMPLES 5 AND 6

Examples 5 and 6 illustrate the ratio of the binary diffusion coefficient of the first component and the sweeping component to the binary diffusion coefficient of the product and the sweeping component (RD) for a process for the preparation of respectively mono-ethylene glycol and mono-propylene glycol. The results are given in table 1.

TABLE 1

| | | | | | Ratio binary |
|---|---|---|---|---|---|
| | | Sweeping | T | P | diffusion |
| Ex. | Mixture | component | (° C.) | (bar) | coefficients (R_{D}) |
| A | H₂O/IPA | CO₂ | 227 | 35 | 2.3 |
| 2 | H₂O/IPA | C3═ | 227 | 35 | 2.5 |
| 3 | H₂O/SBA | C4═ | 34 | 1 | 2.3 |
| 4 | H₂/MEK | SBA | 25 | 1 | 12 |
| 5 | H₂O/MEG | CO₂ | 25 | 1 | 2.6 |
| 6 | H₂O/MPG | CO₂ | 25 | 1 | 2.7 |

1. An integrated separation and preparation process comprising a gas separation process wherein a first component is separated from a feed stream comprising a mixture of components by diffusion of the first component through a porous partition into a stream of sweeping component; and
a preparation process wherein the sweeping component is used as feed.

2. The process of claim 1, wherein the pressure on both sides of the porous partition is essentially equal.

3. The process of claim 1, comprising the steps of
a) gas separating a first component from a mixture of components by diffusion of the first component through a porous partition into a stream of sweeping component to obtain a mixture of first component and sweeping component;
b) optionally separating the mixture of first component and sweeping component obtained in step a) into first component and sweeping component;
c) using the sweeping component, optionally mixed with first component, as a feed to a reaction; and
d) reacting the sweeping component in one or more steps to obtain a product.

4. The process of claim 3, wherein
in step b) the first component and the sweeping component are not separated;
in step c) a mixture of the first component and the sweeping component is used as a feed to a reaction; and
in step d) the first component and the sweeping component are reacted with each other.

5. The process of claim 4, wherein the first component is water, the sweeping component is an alkene; and the first component and the sweeping component are reacted with each other in a hydration reaction to prepare an alkanol.

6. The process of claim 5, wherein the alkene is propene or 2-butene and the alkanol is respectively isopropanol or sec.-butanol.

7. The process of claim 4, wherein the first component is hydrogen, the sweeping component is a ketone; and the first component and the sweeping component are reacted with each other in a hydrogenation reaction to prepare an alkanol.

8. The process of claim 7, wherein the ketone is dimethylketone or methylethylketone and the alkanol respectively is isopropanol or sec.-butanol.

9. The process of claim 4, wherein the first component is hydrogen, the sweeping component is an alkene or an aromatic compound; and the first component and the sweeping component are reacted with each other to prepare an alkane.

10. The process of claim 3, wherein
in step b) the first component and the sweeping component are separated;
in step c) the separated sweeping component is used as a feed in a first reaction and the separated first component is used as a feed in a second reaction; and
in step d) the separated sweeping component is reacted in one or more steps to form a product.

11. The process of claim 10, wherein
in step d) the separated sweeping component is reacted in one or more steps with one or more other components to form an intermediate product; and
the intermediate product is reacted with the separated first component in one or more steps to form a subsequent product.

12. A process comprising the steps of
a) gas separating water from a mixture of water and alkylene glycol by diffusion of the water through a porous partition into a stream of carbon dioxide to obtain a mixture of the water and the carbon dioxide
b) separating the water and the carbon dioxide;
c) using the separated carbon dioxide as a feed in a first reactor and using the separated water as a feed in a second reactor; and
d) reacting the separated carbon dioxide with an alkylene oxide in a first reaction to prepare an alkylene carbonate and reacting the alkylene carbonate with the separated water in a second reaction to prepare an alkylene glycol.

13. The process of claim 12, wherein the alkylene carbonate in step d) is reacted with a surplus of water to prepare a mixture of alkylene glycol and water; and the mixture of alkylene glycol and water is recycled to step a).

14. The process of claim 3, wherein
in step b) the first component and the sweeping component are separated;
in step c) the separated sweeping component is used as a feed to a reaction; and
in step d) the separated sweeping component is reacted in a dehydrogenation reaction.

15. A process comprising the steps of
a) gas separating hydrogen from a mixture of hydrogen and ketone by diffusion of the hydrogen through a porous partition into a stream of alkanol to obtain a mixture of the hydrogen and alkanol;
b) separating the mixture of hydrogen and alkanol into hydrogen and alkanol;
c) using the separated alkanol as a feed in a reaction; and
d) reacting the separated alkanol in a dehydrogenation reaction to obtain a mixture of hydrogen and ketone.

16. The process of claim 15, wherein the mixture of hydrogen and ketone is recycled to step a).

17. The process of claim 15, wherein the alkanol is isopropanol or sec.-butanol and the ketone is respectively dimethylketone or methylethylketone.

18. A separation device for carrying out the gas separation process of claim 1, which comprises a) two or more separation units suitable for gas separating a first component from a mixture of components by diffusion of the first component through a porous partition into a stream of sweeping component; b) a vessel comprising a first fluid inlet opening positioned proximate to a side of the vessel and a first fluid outlet opening positioned proximate to an opposing side of the vessel; and c) a second fluid inlet opening positioned proximate to a side of the vessel and a second fluid outlet opening positioned proximate to an opposing side of the vessel, wherein the first and second inlets and outlets respectively are positioned in such a way that the flow direction of a first fluid stream entering the vessel at the first inlet and leaving it at the first outlet and a second fluid stream entering the vessel at the second inlet and leaving it at the second outlet are essentially perpendicular to each other; and
wherein the porous partition between the two fluids comprises a stack of plate-like structures which are sealed toward the first fluid stream while fluidly connected to the second fluid stream, thereby forming an exterior flow space for the first stream defined at least partially by and positioned at least partially between an upper plate and a lower plate of porous material, and an interior flow space for the second stream, defined at least partially by and positioned at least partially between the opposite sides of the upper plate and the lower plate to prevent fluid flow from the exterior flow space into the interior flow space.

19. A multitubular separation device for carrying out the gas separation process of claim 1, comprising
a substantially vertically extending vessel;
a plurality of tubes having a porous wall arranged in the vessel parallel to its central longitudinal axis of which the upper ends of the tubes are fixed to an upper tube plate and in fluid communication with a top fluid chamber above the upper tube plate and of which the lower ends are fixed to a lower tube plate and in fluid communication with a bottom fluid chamber below the lower tube plate, wherein the porous wall is suitable for gas separating a first component from a mixture of components by diffusion of the first component through such porous wall into a stream of sweeping component;
supply means for supplying a first fluid to the top fluid chamber;
an effluent outlet arranged in the bottom fluid chamber;
supply means for supplying a second fluid to the space between the upper tube plate, the lower tube plate, the outer surface of the tubes and the vessel wall; and
an effluent outlet from such space between the outer surface of the tubes and the vessel wall.

20. The separation device of claim 18, further comprising a pressure balancing means to maintain the pressures at each side of the porous partition essentially equal.

21. An industrial apparatus for carrying out the process of claim 1, comprising
a separation device comprising one or more separation units suitable for gas separating a first component from a mixture of components by diffusion of the first component through a porous partition into a stream of sweeping component, said device comprising one or more first chambers, one or more second chambers which are separated from the first chamber or chambers by a porous partition, one or more inlets and one or more outlets; and
one or more reactors comprising one or more inlets and one or more outlets, wherein the outlet of said one or more separation units is connected directly or indirectly to said one or more inlets of one or more reactors.

22. (canceled)

23. The industrial apparatus of claim 21, wherein the separation device comprises two or more separation units suitable for gas separating a first component from a mixture of components by diffusion of the first component through a porous partition into a stream of sweeping component, wherein each separation unit comprises
a first chamber;
a second chamber, separated from the first chamber by a porous partition;
a first inlet for conveying a mixture of components to the first chamber;
a first outlet for discharging the remainder of the mixture of components after at least part of the first component has been removed from the first chamber;
a second inlet for conveying a sweeping component into the second chamber; and
a second outlet for discharging a mixture of sweeping component and diffused first component from the second chamber.

24. The separation device of claim 19, further comprising a pressure balancing means to maintain the pressures at each side of the porous partition essentially equal.
